(19) United States
(12) Patent Application Publication
(10) Pub. No.: US 2018/0160971 A1
(43) Pub. Date: Jun. 14, 2018

(54) STEREORADIOGRAPHY MEASUREMENT OF ARTHROPLASTY IMPLANT LOOSENING

(71) Applicant: HALIFAX BIOMEDICAL INC., Mabou (CA)

(72) Inventors: Johan Erik Giphart, Mabou (CA); Chad Munro, Mabou (CA)

(73) Assignee: HALIFAX BIOMEDICAL INC., Mabou, NS (CA)

(21) Appl. No.: 15/580,727

(22) PCT Filed: Jun. 10, 2016
(86) PCT No.: PCT/CA2016/050661

§ 371 Date: Dec. 8, 2017

Related U.S. Application Data
(60) Provisional application No. 62/173,573, filed on Jun. 10, 2015.

(51) Int. Cl. A61B 5/00; A61B 6/02; A61B 6/00; A61F 2/46

(52) U.S. Cl. CPC A61B 5/4851; A61B 6/022; A61B 2090/3966; A61F 2/468; A61B 6/505

(57) ABSTRACT

The present invention relates to a device and use of the device for detection and loosening of implants installed into a subject's host bone, the device comprising: a frame for encircling and demountably engaging a portion of the subject's appendage comprising the host bone with installed implant; and at least three-spaced apart attachments engaged with the frame, wherein each attachment is configured to house and to retractably deploy a sharp geometry component for contacting the host bone, wherein a temporary reference frame is created when the three-spaced apart attachments contact the host bone. The use of the device comprises obtaining a first pair of stereo radiographs of the implant under a first loading condition and obtaining a second pair of stereo radiographs of the implant and host bone area under the second loading condition and comparing the two radiographs to detect displacement of the implant.

### FIELD OF THE INVENTION

The present invention relates to stereo radiographic assessments of implant loosening and to diagnostic methods for diagnosing implant loosening.

### BACKGROUND OF THE INVENTION

During their lifetime, one in two people will develop symptomatic knee osteoarthritis and one in four will develop symptomatic hip osteoarthritis. When symptoms become too severe and the osteoarthritic process reaches its end stages, total joint replacement (arthroplasty) is a well-established and generally successful treatment option. The number of hip and knee joint replacements is expected to increase significantly over the next decades to approximately 1.0 million hip replacements and 4.3 million knee replacements annually by 2030. However, 5-10% of patients who received a joint replacement will require a revision surgery within 10 years of the index surgery. A major cause of hip and knee replacement failure and subsequent revision is aseptic loosening.

Gross loosening of implants is visible on conventional x-rays and may take several years to develop. However, the earlier stages of implant loosening involve very subtle sub-millimeter movements or migration of the implant relative to the host bone. Such small initial movements cannot be detected with conventional methods. Stereo orthopaedic radiography (also known as Roentgen Stereophotogrammetric Analysis, Radio Stereometric Analysis, or RSA) is a measurement methodology designed to measure early implant loosening. This methodology requires the implantation of at least three radiopaque markers (typically 1.0-mm diameter tantalum balls) into the host bone during the arthroplasty procedure to serve as an accurate reference frame for measurement of the implant's migration. Following the index surgery and marker implantation, a series of stereo orthopaedic radiography (SOR) images are taken over time consisting of two x-ray images taken at the same time from different angles and with overlapping beams such that a triangulation method for measurement reconstruction is possible. Software is used to analyze these image pairs to assess the implant's position relative to the host bone. Assessing these positions at multiple time points enables generation of implant migration curves in multiple dimensions. Such migration curves have been demonstrated to predict implant loosening.

At least three radiopaque markers are required for implantation into the bone for precise three-dimensional imaging of bone position and for detection and assessments of implant loosening. There is often an insufficient number of markers present, or no markers present, to perform the measurements required to detect a loose implant.

### SUMMARY OF THE INVENTION

The embodiments of the present disclosure relate to devices and methods for use in assessing implant loosening. Specifically, the exemplary embodiments of the present disclosure pertain to patients who did not have markers implanted in the host bone of their joint replacement or other implant of interest at the time of installation surgery.

Some embodiments of the present disclosure may relate to patients who did not have a sufficient number of markers implanted in their host bone or alternatively, an insufficient number of markers visible in the x-ray images to allow precision measurements.

Rather than surgical implantation of markers into the bone post index-surgery, the exemplary embodiments of the present disclosure comprise a device that may be securely attached around a patient's limb and secured to a patient's bone in a minimally invasive manner for the duration of an assessment episode, and which can subsequently be removed once the assessment has been completed. The device is securely attached to the host bone by applying sterile sharp geometry components exemplified by pins or needles, connected to a frame and through the skin to make direct contact with the bone using suitable sterile procedures and under local anaesthetic when necessary. Suitable sharp geometry components are exemplified by cannulated or solid sharp objects that can be inserted through the skin to contact the underlying bone and which will not slide on the bone surface once in contact with the bone. Such suitable geometry components are exemplified by needles such as injection needles and biopsy needles, wires such as Kirschner wires, and pins such as Steinmann pins, and the like.

According to one embodiment of the present device, the frame component of the device contains radiopaque markers. According to another exemplary embodiment, the sharp geometry components of the device contain radiopaque markers. According to another exemplary embodiment, the assessment method uses the tips of the sharp geometry components, or alternatively, the shapes of the sharp geometry components, or alternatively, other unique marker features associated with the sharp geometry components to establish suitable marker reference points relative to the host bone to measurements of implant motion or migration relative to host bone. According to another exemplary embodiment of the invention, the needle components may contact the host bone and/or the implant surfaces, or alternatively, may penetrate the host bone.

Some embodiments of the present invention comprise methods for measuring implant loosening with the devices disclosed herein. The methods generally comprise the steps of: (i) obtaining at least two sets of stereo orthopaedic radiographs of a selected host bone and implant during engagement with the external marker device attached to a bone within the subject's appendage during at least two different loading conditions designed to displace a loose implant relative to its host bone, (ii) assessing the implant position relative to the temporary reference provided by the external marker device in each loading condition, and (iii) calculating the amount of implant motion between the two or more loading conditions. Displacements above a certain threshold exemplified by being a translation, a rotation, and/or a maximum total point motion, are considered indicative of a loose implant. Maximum total point motion is the amount of motion of the point on an implant which moved the most. Persons of skill in the art will recognize that there are a variety of more advanced benchmarks that can be developed to be indicative of implant loosening without limiting the foregoing. Persons of skill in the art will recognize that the same method can be followed using single plane x-ray imaging (e.g., single plane radiology or fluoroscopy) instead of stereo orthopaedic radiographs at the expense of possibly losing out-of-plane precision and accuracy without limiting the foregoing.

### BRIEF DESCRIPTION OF THE DRAWINGS

These and other features of the invention will become apparent in the following detailed description in which reference is made to the appended drawings.

FIG. 1 is a conceptual illustration of a knee joint area of a tibia bone and fibula bone adjacent to an implant prior to installation of the implant;

FIG. 2 is a conceptual illustration of the device attached to the tibia bone, according to embodiments of the present disclosure;

FIG. 3 is a conceptual illustration of the mechanism used to maintain contact between the bone and a sharp geometry component according to embodiments of the present disclosure;

FIG. 4 is a schematic illustration of a stereo orthopaedic radiography imaging system; and

FIG. 5 is a schematic illustration of several loading conditions for the knee.

### DETAILED DESCRIPTION OF THE INVENTION

Aseptic loosening is a common cause for revision in joint replacement surgery and is difficult to diagnose. Not until there is substantial loosening are radiolucent lines visible around the implant on standard radiographs. Radio stereometric analysis of stereo orthopaedic radiography images is a very accurate measurement technique able to measure precise 3D location of implants and host bones. Assessing these positions at multiple time points enables production of implant migration curves in multiple dimensions. Such early migration measurements have been shown to be able to predict aseptic loosening. However, the standard technique is based on the implantation of tantalum markers into a patient's host bone at the time of the joint replacement surgery for the purpose of providing a reference frame for 3D positioning and migration measurements. If these markers are not implanted at the time of surgery, the migration measurements cannot be made due to the lack of an accurate reference frame. Post-operative implantation of markers is possible but carries significant additional clinical risk if not done in an OR setting and therefore, is not clinically practical.

The embodiments of the present disclosure describe devices and methods that allow for the assessment of implant loosening without the requirement to have markers permanently implanted into the host bone. Specifically, the embodiments of the present disclosure provide a set of temporary reference points for accurate implant loosening measurement using at least three or more spaced-apart sharp geometry components that temporarily contact the bone. The sharp geometry components are housed within holders that are disposed about a frame that is temporarily de-attachably mountable around a subject's joint area of a host bone that houses an installed implant.

Some exemplary embodiments of the present disclosure pertain to methods of imaging the frame, the sharp geometry components and implant using a stereo orthopaedic radiography system under two or more loading conditions aimed at loading the implant of interest such that a loose implant moves (migrates) relative to the host bone, and thus, relative to the temporary reference frame when it is temporarily secured in place against the host bone.

### Definitions

Unless defined otherwise, all technical and scientific terms used herein have the same meaning as commonly understood by one of ordinary skill in the art to which the invention belongs.

As used herein, the terms “x-ray” and “radiographic imaging” are used interchangeably through the application to mean the use of electromagnetic radiation to view the internal skeletal structures within a mammalian subject's body.

As used herein, the term “about” refers to an approximately +/−10% variation from a given value. It is to be understood that such a variation is always included in any given value provided, whether or not it is specifically referred to.

As used herein, the term “sharp geometry component” refers to a cannulated or solid sharp contact geometry that can be inserted into and through the skin to contact the underlying bone, and which will not move relative to the bone once in contact with the bone and secured to a patient's appendage.

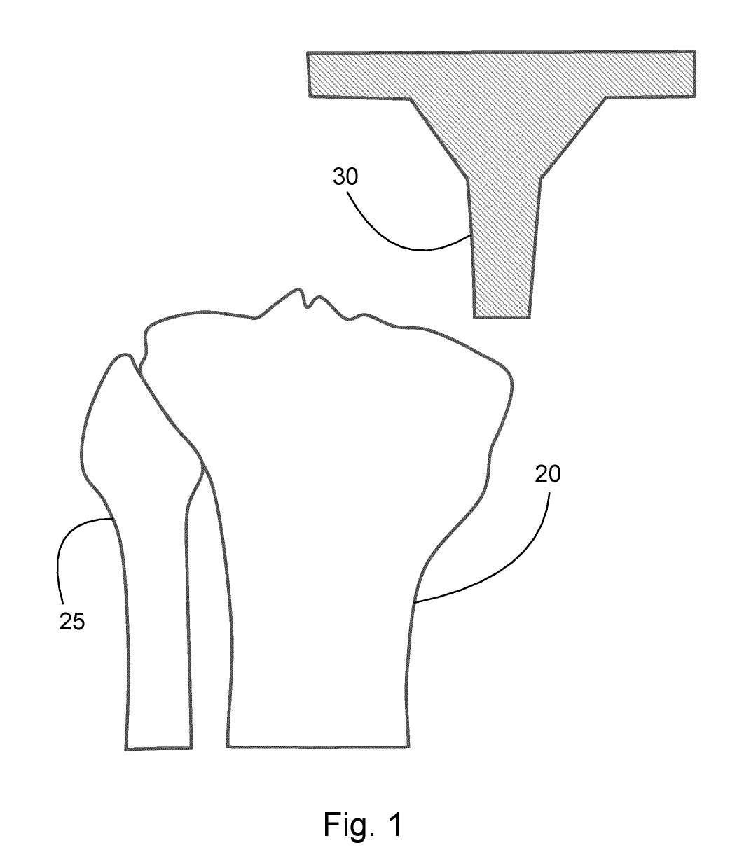

For purposes of illustration, the devices and methods of the invention are described below with reference to the knee of the human body. However, as will be appreciated by those skilled in the art, the devices and methods can be employed with any mammal and for any joint wherein an implant has been securely installed. Exemplary embodiments of the present disclosure will now be described by reference to FIGS. 1 to 5.

### Temporary Reference Device for Providing a Temporary Reference Frame

Persons of skill in the art will recognize that there are a variety of devices that may be used to place at least three or more sharp geometry components in contact with the bone around a joint replacement, or other type of, implant for the purpose of measuring implant loosening. Some exemplary embodiments of the present disclosure relate to a device comprising of a frame for encircling and engaging a portion of a subject's appendage with the host bone and installed implant (fore example, a knee joint), at least three attachments engaged and cooperating with the frame wherein each attachment is configured to retain a sharp geometry component and to apply a small load to a sharp geometry component, and at least three sterile sharp geometry components able to penetrate the skin and underlying soft tissues and to touch the bone without significantly penetrating the bone. It is within the scope of the present disclosure for the frame and/or the attachments to comprise a rigid material, a semi-rigid material, or a soft material. According to some aspects, more than three pins and/or needles may be used for contacting the subject's target bone. According to some aspects, the frame may be secured in place about the appendage with the target joint with one or more straps, belts, bands, or other type of securing mechanism. Alternatively, the frame may be placed into a harness for securing to the subject's appendage.

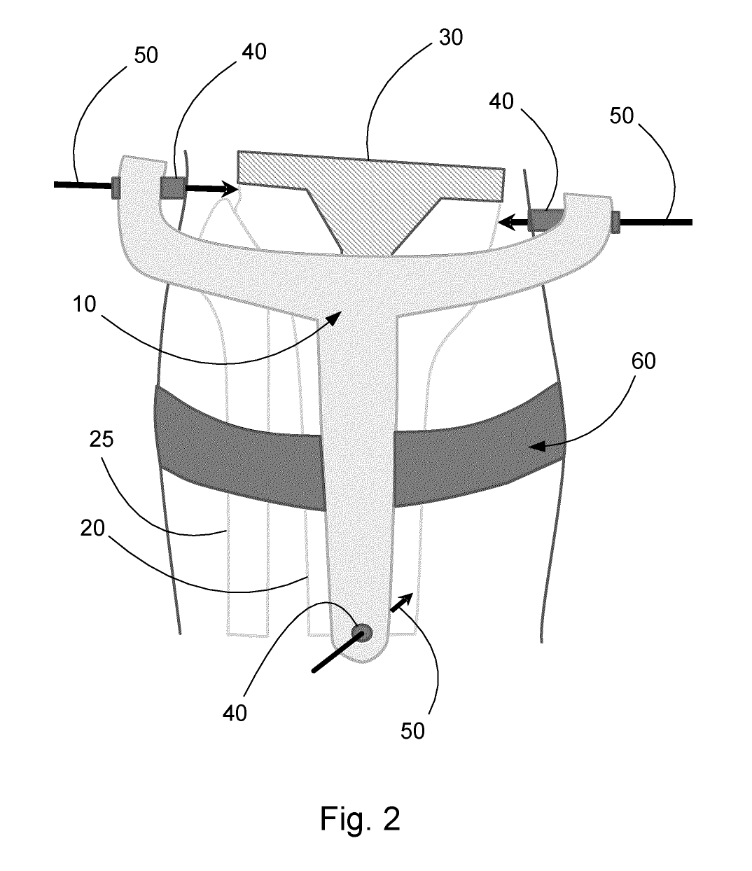

FIG. 1 illustrates the knee joint area with an implant 30 to be installed into the tibia 30 (the fibula 25 is shown for reference). As illustrated in FIG. 2, an exemplary device of the present disclosure comprises a frame 10 positioned over the tibia 20 into which a tibial component 30 of a knee joint replacement has been installed. In this exemplary embodiment, three attachment mechanisms 40 are attached to frame 10 wherein each of the attachment mechanisms 40 is provided with a retractably extendible sharp geometry component 50 for contacting the tibia bone 20. The sharp geometry components are sterile so as to avoid infection and are selected for easy entry into and through the skin and underlying soft tissue, but which will not: (i) significantly penetrate the cortical bone, and (ii) easily slide over the bone surface when a sheer load is applied. These sharp geometry components are engaged with a patient's appendage and host bone using proper sterile methods. It is critical for the invention that the tips of the sharp geometry components do not change location for the duration of the measurement. According to further embodiments of the current disclosure, the sharp geometry components may also be inserted through a small stab incision. One or more straps 60 may optionally be provided to secure the frame to the subject's appendage, in this case a knee joint area, to keep the frame in place during initial set-up and/or during the imaging procedure during which the patient may be required to move.

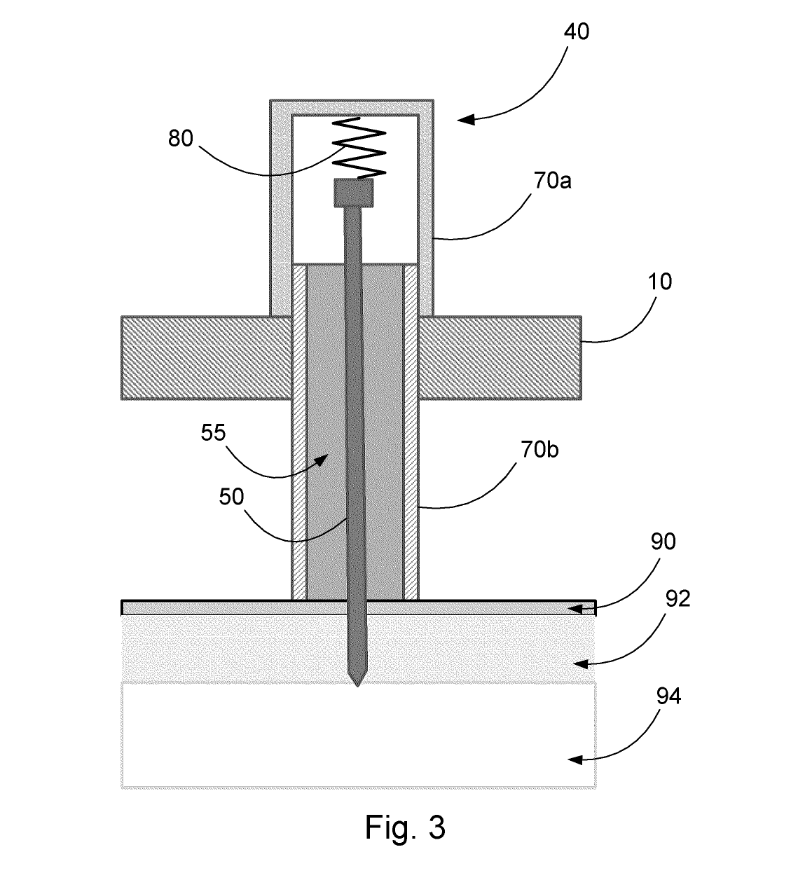

An exemplary attachment mechanism 40 is illustrated in FIG. 3. The attachment mechanism 40 is attached to the frame 10 with a two-piece adjustable housing 70a, 70b which may or may not extend from the frame down to the skin. The sharp geometry component 50 is securely mounted in a holder 55 provided in the housing 70b and held in place relative to frame 10. The housing 70a is provided with a loading mechanism 80 exemplified by a spring, which is used to apply a slight force to the sharp geometry component 50 such that the sharp geometry component 50 stays in firm contact with the underlying bone 90.

### Measurement of Implant Loosening

Migration of an implant relative to its host bone can be measured accurately using stereo orthopaedic radiography. Persons of skill in the art will recognize that there are a variety of devices that may be used to obtain simultaneous x-ray images of an implant taken using two x-ray systems and from two different vantage points (i.e., stereo orthopaedic images). In addition, persons of skill in the art will recognize that two sequential images using one or two x-ray systems may be used to obtain x-ray images of an implant taken from two different vantage points which under appropriate conditions may also constitute stereo orthopaedic images. Persons of skill in the art will recognize that the same method can be followed using single plane x-ray imaging (e.g., single plane radiology or fluoroscopy) instead of stereo orthopaedic radiographs at the expense of possibly losing out-of-plane precision and accuracy without limiting the foregoing. Some exemplary embodiments of the present disclosure relate to a method for detecting and assessing migration of an installed implant wherein the method comprises the steps of securing the device around a subject's joint of interest so that each of the sharp geometry component holders is positioned about a target location on the host bone, inserting each of the sharp geometry components through the subject's skin surface and soft tissue until the tip of the sharp geometry component touches the host bone surface, obtaining a first pair of stereo radiographs of the implant and host bone area under a first loading condition, placing a second load on the joint, obtaining a second pair of stereo radiographs of the implant and host bone area under the loaded condition, comparing the first pair of stereo radiographs and the second pair of stereo radiographs, detecting if the implant was displaced in the second loaded condition, if a displacement was detected, determining the distance the implant was displaced in the second loaded condition, and determine if the displacement distance is indicative of a loosened implant or not. It is to be noted that a displacement may be translational or rotational and in may occur in one or more dimensions.

Persons of skill in the art will recognize that there are a variety of methods that may be used to apply a load to an implant directly or indirectly in an attempt to induce motion of the implant relative to the host bone when the implant is loose. Without limiting the foregoing, certain embodiments of the present disclosure may load or unload the joint which contains the implant of interest by laying down on a table, by bearing weight or partial weight on the limb containing the implant or both limbs, by applying a rotatory moment to the joint or limb, by applying weights or force directly to the joint, etc.

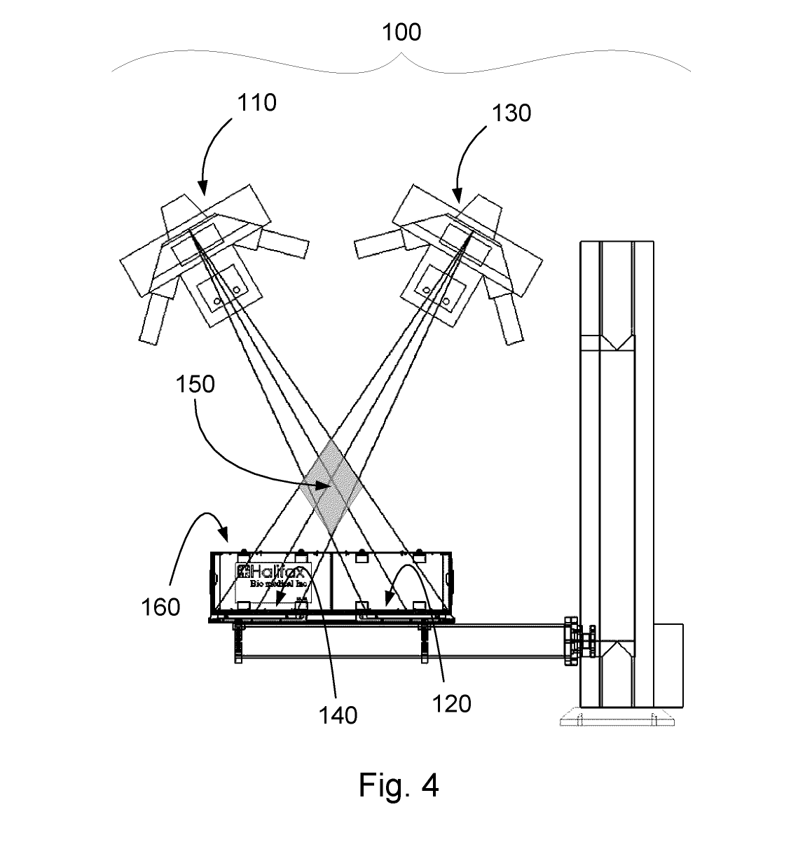

Referring to FIG. 4, an exemplary stereo radiography system 100 is illustrated. An x-ray source 110 is aimed at an x-ray detector 120 at an angle from vertical. In addition, a second x-ray source 130 is aimed at an x-ray detector 140 such that the x-ray beams overlap in the 3D viewing area 150. As long as the removable reference frame and implant are placed in the 3D viewing area under the various loading conditions the accurate measurement of implant displacement relative to the reference frame can be made. The stereo radiography system 100 may or may not include a reference box 160 containing fiducial and control markers to aid in accurately determining the 3D x-ray configuration.

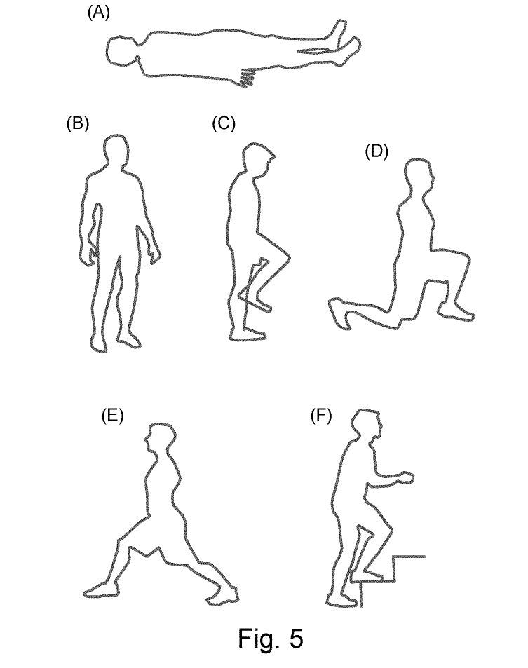

FIGS. 5(A)-5(F) illustrates suitable loading conditions for a knee joint for assessment with an exemplary method disclosed herein, exemplified by (A) unloading by lying down (supine), (B) full weight bearing during standing, (C) partial weight bearing during standing, (D) full lunge position, (E) partial lunge position, (F) stair stepping positions.

1. A device for detection of and assessment of loosening of implants installed into a subject's host bone, the device comprising:
a frame for encircling and demountably engaging a portion of the subject's appendage comprising the host bone with installed implant; and
at least three-spaced apart attachments engaged with the frame, wherein each attachment is configured to house and to retractable deploy a sharp geometry component for contacting the host bone,
wherein a temporary reference frame is created when the three-spaced apart attachments contact the host bone.

2. A device according to claim 1, additionally comprising at least one strap cooperative with the frame for demountably engaging the subject's appendage.

3. A method for detection of and assessment of loosening of implants installed into a host bone, the method comprising:
securing the device of claim 1 around a subject's joint of interest so that each of the attachments is positioned about a target location on the host bone;
inserting each of the sharp geometry components through the subject's skin surface and soft tissue until the tip of each sharp geometry component contacts the host bone surface;
obtaining a first pair of stereo radiographs of the implant and host bone area under a first loading condition;
placing a second load on the joint;
obtaining a second pair of stereo radiographs of the implant and host bone area under the second loading condition;
comparing the first pair of stereo radiographs and the second pair of stereo radiographs;
detecting if the implant is displaced in the second loading condition;
if a displacement is detected, determining the distance the implant was displaced in the second loading condition; and
determining if the displacement distance is indicative of a loosened condition.
